(19) United States
(12) Patent Application Publication
(10) Pub. No.: US 2004/0191776 A1
(43) Pub. Date: Sep. 30, 2004

(54) Method for genotyping and quantifying Hepatitis B Virus

(76) Inventors: Pei-Jer Chen, Taipei (TW); Ding-Shinn Chen, Taipei (TW); Shiou-Hwei Yeh, Taipei (TW); Tsong-Chin Lin, Hsin Chu (TW)

Correspondence Address: FISH & RICHARDSON PC, 225 FRANKLIN ST, BOSTON, MA, 02110, US

(21) Appl. No.: 10/395,013

(22) Filed: Mar. 21, 2003

(51) Int. Cl. C12Q 1/70; C07H 21/04; C12Q 1/68

(52) U.S. Cl. 435/6; 435/5; 536/23.72; 536/24.3; 536/24.32; 536/24.33

(57) ABSTRACT

A method for simultaneously genotyping and quantifying Hepatitis B Virus. Also disclosed are (1) a pair of primers containing, respectively, the sequences of SEQ ID NOs: 13 and 14, SEQ ID NOs: 17 and 14, or SEQ ID NOs: 20 and 6, each primer being 8-50 nucleotides in length; (2) a pair of probes, containing, respectively, the sequences of SEQ ID NOs: 18 and 19, SEQ ID NOs: 15 and 16, or SEQ ID NOs: 21 and 22, each probe being 9-50 nucleotides in length; (3) a nucleic acid obtained from amplification of a Hepatitis B Virus nucleic acid template, containing the sequence selected from SEQ ID NOs: 15, 19, or 22, or its complementary sequence, the nucleic acid being 100-1,000 nucleotides in length.

### BACKGROUND

Single nucleotide polymorphisms (SNPs), a set of single nucleotide variants at genomic loci, are distributed throughout a genome. A single nucleotide polymorphism can be “allelic.” That is, due to the existence of the polymorphism, some members of a species may have the unmutated sequence (i.e. the wild-type allele) whereas other members may have a mutated sequence (i.e. the mutant allele). In animals, a polymorphism may cause genetic recessive disorders. These disorder include bovine leukocyte adhesion deficiency, citrullinemia, maple syrup urine disease, deficiency of uridine monophosphate synthase, a-mannosidosis, and generalized glycogenosis. In humans, an example of genetic recessive disorders is cystic fibrosis, which affects about 1/2000 individuals of the entire Caucasian population. In microbial pathogens, such as bacteria and viruses, single nucleotide polymorphisms are associated with different pathological effects, and therefore have bearing on therapy of, and long-term prognosis for, patients infected with the pathogens. A method is in need for efficiently identifying and quantifying a SNP-containing nucleic acid.

### SUMMARY

This invention relates novel primers, probes, and nucleic acids amplified from Hepatitis B virus (HBV) and their use in simultaneously genotyping and quantifying HBV.

In one aspect, the invention relates a method for simultaneously identifying a SNP in a target nucleic acid (e.g., as the entirety or part of a genome) from an HBV and quantifying the target nucleic acid. The identification and quantification are carried out simultaneously.

This method requires the use of a first probe (or sensor probe) and a second probe (or anchor probe). The first probe is identical or complementary to a first sequence of the target nucleic acid that covers a base corresponding to a SNP. The second probe is identical or complementary to a second sequence of the target nucleic acid that does not cover the base corresponding to the SNP. The first probe is covalently bounded to a first fluorophore, and the second probe is covalently bounded to a second fluorophore. One of the first and second fluorophores is a donor fluorophore, and the other is an acceptor fluorophore, so that, when the first probe and the second probe are hybridized to the target nucleic acid, the donor fluorophore and the acceptor fluorophore are in close proximity to allow fluorescence resonance energy transfer (FRET) between them.

The method includes amplifying, by a polymerase chain reaction (PCR) with a pair of primers, a target nucleic acid in a sample to form a double-stranded nucleic acid product containing the first sequence and the second sequence. The above-mentioned first and second probes are hybridized to the nucleic acid product during the annealing step of the PCR to form a first duplex and a second duplex, respectively. The two probes can be hybridized to the same strand of the nucleic acid product. They can also be hybridized to different strands of the nucleic acid product and allow the donor and acceptor fluorophores to be in close proximity. For example, the two probes can be hybridized to sequences at a fork or bubble formed by the two strands of the nucleic acid product.

The target nucleic acid in a sample is quantified by monitoring the fluorescent emission of the acceptor fluorophore on the first probe at the end of the annealing phase of each PCR cycle. It can be achieved by comparing the intensity of the fluorescent emission with a value predetermined from a solution containing a known concentration of the target nucleic acid. It can also be achieved by obtaining a cross point value (Cp value) of the PCR reaction and comparing the Cp value to another Cp value predetermined from a solution containing a known concentration of the target nucleic acid by the method described in, e.g., Mackay I. et al., Nucleic Acids Res. 30: 1292-1305, 2002.

After the PCR reaction, the temperature is raised to above the melting temperature of the duplex formed by the first probe and its complementary sequence. When this duplex is dissociated, the FRET between the donor and acceptor fluorophores is disrupted. Identification of a SNP in the target nucleic acid is achieved by monitoring fluorescent emission change of the acceptor fluorophore on the first probe upon irradiation of the donor fluorophore with an excitation light, the change being a function of the elevated temperature. For example, to identify a SNP, one can (1) generate a first derivative melting curve of the first duplex, which includes a fluorescently labeled probe, based on fluorescent emission change as a function of temperature; (2) determine a temperature value corresponding to a melting peak on the curve; and (3) compare the temperature value with the melting temperature of the duplex formed by the first probe and its complementary sequence. A SNP in the target nucleic acid is present when the temperature value is lower than the melting temperature and is absent when the temperature value is the same as the melting temperature.

In another aspect, the invention features a pair of primers for amplifying a target nucleic acid of HBV, such as forward primer TACTGCGG (SEQ ID NO: 13) and reverse primer GGTGAAGCGA (SEQ ID NO: 14); forward primer CGTGGAACC (SEQ ID NO: 17) and reverse primer GGTGAAGCGA (SEQ ID NO: 14); or forward primer CTCAGGCCA (SEQ ID NO: 20) and reverse primer AACGCCGCAGACACATCCA (SEQ ID NO: 6). Each primer is 8-50 nucleotides (e.g., 15-40 or 18-30 nucleotides) in length. More examples of such a pair of primers include forward primer CCGATCCATACTGCGGAAC (SEQ ID NO: 9) and reverse primer GCAGAGGTGAAGCGAAGTGCA (SEQ ID NO: 10); forward primer GCATGCGTGGAACCTTTGTG (SEQ ID NO: 1) and reverse primer CAGAGGTGAAGCGAAGTGC (SEQ ID NO: 2); and forward primer TCATCCTCAGGCCATGCA (SEQ ID NO: 5) and reverse primer AACGCCGCAGACACATCCA (SEQ ID NO: 6).

In still another aspect, the invention features a pair of probes used together for identifying a SNP in a target nucleic acid of HBV and quantifying the target nucleic acid, such as a first probe TTGTCTACG (SEQ ID NO: 18) and a second probe CGCTGAATC (SEQ ID NO: 19); sensor probe TACGCGGACTC (SEQ ID NO: 15) and anchor probe GCCTTCTCATC (SEQ ID NO: 16); or sensor probe ACACGGGTGTTTCC (SEQ ID NO: 21) and anchor probe ATTGAGAGAA (SEQ ID NO: 22). Each probe is 9-50 nucleotides in length, e.g., 15-40 or 18-30 nucleotides in length. More examples of such a pair of probes include sensor probe ACGTCCTTTGTCTACGTCCCG (SEQ ID NO: 3) and anchor probe CGGCGCTGAATCCCGCGGAC (SEQ ID NO: 4); sensor probe TCTTTACGCGGACTCCCC (SEQ ID NO: 1) and anchor probe TCTGTGCCTTCTCATCTGCCGGACC (SEQ ID NO: 12); and sensor probe AAGACACACGGGTGTTTCCCC (SEQ ID NO: 7) and anchor probe GAAAATTGAGAGAAGTCCACCACGAGTCTA (SEQ ID NO: 8).

In yet another aspect, the invention features a nucleic acid that is obtained from amplification of a HBV nucleic acid template. This nucleic acid contains the sequence of SEQ ID NO: 15, 18, or 21, or its complementary sequence. The nucleic acid is 100-1,000 (e.g., 200-700 or 300-500) nucleotides in length. This nucleic acid can be hybridized with the above-mentioned probes and primers for identifying and quantifying a SNP-containing target nucleic acid of HBV.

In a further aspect, the invention features a kit for simultaneously identifying a single nucleotide polymorphism in a target nucleic acid of HBV and quantifying the target nucleic acid. The kit contains 1, 2, or 3 pairs of the primes and/or probes described above.

The details of one or more embodiments of the invention are set forth in the accompanying description below. Other advantages, features, and objects of the invention will be apparent from the detailed description and the claims.

### DETAILED DESCRIPTION

The present invention relates to a method for simultaneously genotyping and quantifying Hepatitis B Virus. This method requires the use of a first probe and a second probe. The first probe can be designed based on a known SNP in a target nucleic acid, and also based on its properties, e.g., GC-content, annealing temperature, or internal pairing, which can be determined using software programs. For identifying a SNP in nucleic acids from different members of a species, the first probe should be identical or complementary to a sequence containing a SNP that can be used to distinguish between at least two different genotypes of the species. Such a sequence can be determined based on standard sequence alignment of the DNA from different members of the species in a manner similar to that described below in the “Design of probes and primers” section. The DNA sequences of different members can be obtained from any suitable databases, e.g., www.ncbi.nlm.nih.gov/PMGifs/Genomes.

The first probe can hybridize to one SNP allele, e.g., a wild-type allele, to form a duplex with no mismatched base, and to another SNP allele, e.g., a mutant allele, to form another duplex with mismatched bases at the SNP site(s). Due to the mismatches, the melting temperature (Tm) of the latter duplex is lower that that of the former. The first probe can be optimized on a gene-by-gene basis to discriminate between a wild-type allele and a mutant allele. One can confirm empirically the ability of the first probe to hybridize to a mutant allele or a wild-type allele to form duplexes. One can also confirm the difference between the melting temperatures of the two duplexes are sufficiently great (e.g., 2° C.) so that the difference can be detected.

SNP-containing sequences in HBV include TACGCGGACTC (SEQ ID NO: 15), TTGTCTACG (SEQ ID NO: 18) and ACACGGGTGTTTCC (SEQ ID NO: 21). (The bases corresponding to SNPs are bold and underlined). These SNPs can be used to distinguish HBV genotypes A to G. See Tables 1 and 2, and the “Simultaneous quantification and identification of HBV” section below.

The SNP-containing sequences are preferably flanked by sequences that are conserved among different genotypes of a species. As described below, the conserved flanking sequences are important for designing a second probe and PCR primer pairs.

The second probe is designed based on two principles. First, it contains no SNP and is identical or complementary to a sequence conserved among different genotypes of a species. Second, the conserved sequence should be adjacent to the above-described SNP-containing sequence. This is to ensure that, after the first and the second probes hybridize to a target nucleic acid, the two probes are in close proximity, e.g., 1-3 bases apart.

Each of the first and second probes is labeled with a fluorophore that can be detected, directly or indirectly, by techniques well known in the art. One of the fluorophore is an acceptor fluorophore, and the other is a donor fluorophore. The emission spectrum of the donor fluorophore overlaps the excitation spectrum of the acceptor fluorophore. The donor and acceptor fluorophores are so located that, upon hybridization of the probes to a target nucleic acid, they are within a short distance of each other to allow FRET to takes place between them. The emission of the acceptor can be detected and/or quantified by techniques well known in the art. Any pair of fluorophores that having overlapped emission and excitation spectra can be labeled to the two probes. LightCycler-Red 640 is an example of an acceptor fluorophore, and fluorescein is an example of a donor fluorophore.

To simultaneously quantify and identify a target nucleic acid, the above-described probes are mixed with the target nucleic acid and subjected to a Real-time PCR reaction. A pair of primers used for the PCR can be designed based on principles known in the art. In particular, the primers should be identical or complementary to sequences that flank a SNP and are conserved among different genotypes of a species. The pair of primers can be used to amplify a target nucleic acid contained a SNP. The nucleic acid can be obtained from any suitable source, e.g., a tissue homogenate, blood samples and can be DNA or RNA (in the case of RNA, reverse transcription is required before PCR amplification). PCR amplification can be carried out following standard procedures. See, e.g., Innis et al. (1990) PCR Protocols: A Guide to Methods and Applications Academic Press, Harcourt Brace Javanovich, New York. In one example, Real-time PCR amplification was carried out using a commercially available Real-PCR system (e.g., LightCycler marketed by Roche Molecular Diagnostic.).

The 3 steps of PCR amplification denaturing, annealing and elongating, can be repeated as many times as needed to produce the desired quantity of an amplification product corresponding to the target nucleic acid. The required cycling number depends on, among others, the nature of the sample. If the sample is a complex mixture of nucleic acids, more cycling steps will be required to amplify the target sequence sufficient for detection. Generally, the cycling steps are repeated at least about 20 times, but may be repeated as many as 40, 50, 60, or even 100 times. The PCR product can anneal with the above-described probes and is used to identify and quantify the target nucleic acid.

To quantify a target nucleic acid, fluorescence emitted from the acceptor fluorophore is monitored at the end of the annealing phase of each PCR cycle upon irradiation of the donor fluorophore. The intensity of the fluorescence emission is a function of the amount of the amplified nuclei acid product, which, in turn, is a function of the original concentration of the target nucleic acid and PCR cycle numbers. When enough cycles are carried out, the rates for the accumulation of the amplified nuclei acid product and for the change in the fluorescence emission enter a log-linear phase. The PCR cycle number corresponding to the entry point (the cross point value, or Cp value) can be determined by plotting the fluorescence emission intensity against the PCR cycling number. The Cp value thus obtained is then compared to a predetermined Cp value that corresponds to a known original concentration of a standard nucleic acid. A series of such predetermined Cp values can be obtained in the manner described below in the “Quantification of HBV” section. Accordingly, one can derive the corresponding original concentration of the target nucleic acid by comparing a given Cp value to a series of predetermined Cp values.

Alternatively, one can simply compare the emission intensity with a predetermined emission intensity value to quantify a target nucleic acid. The predetermined emission intensity value is acquired in the same manner except that the original concentration of nucleic acid is known.

To identify a target nucleic acid, its amplicon is subjected to a melting curve analysis at the end of the PCR amplification. The reaction is heated slowly, e.g., at a transition rate of 0.5° C./sec, to a temperature higher than the Tm of the first probe. Meanwhile, fluorescence emitted from the acceptor fluorophore is monitored upon irradiation of the donor fluorophore. The intensities (F) are plotted against temperature (T) to generate a melting curve. Then, a first derivative of the melting curve (i.e., a first derivative melting curve) is generated by plotting the negative derivative of F with respect to temperature (−dF/dT) against T to located a melting peak(s). The temperature value corresponding to the melting peak is then compared with the melting temperature of the first probe. In a preferred embodiment, the melting curve analysis is performed using LightCycler analysis software 3.5 (Roche Diagnostics Applied Science, Mannheim Germany). A SNP in the target nucleic acid is present when the temperature value is lower than the melting temperature and is absent when the temperature value is the same as the melting temperature. Unexpectedly, the method described herein is very efficient as it simultaneously quantifies and identifies a SNP-containing nucleic acid.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

Design of Probes and Primers

216 full-length HBV DNA sequences were obtained from the database at www.ncbi.nlm.nih.gov/PMGifs/Genomes/viruses.html. Among them, 175 sequences were identified as belonging to genotypes A to G, by a phylogenic analysis using the CLUSTRLW Multiple Sequence Alignment, DRAWTREE, and DRAWGRAM programs provided by Biology WorkBench (workbench.sdsc.edu/).

Among the 175 sequences, 47 were genotype B, and 49 were genotype C. The sequences of the two genotypes were aligned and compared to identify regions that contain SNPs and are flanked by sequences conserved between the two genotypes by the CLUSTRLW Multiple Sequence Alignment program. Three regions were identified, and three sets of primer pairs and probe pairs were designed based on the sequences of the regions according to principles suggested by TIB MOLBIOL (Gerlin, Germany). The primer pairs can be used, via PCR, to prepare amplicons from respective target nucleic acids. The genomic locations of the amplicons, primer pairs, and probe pairs are summarized in Table 1.

TABLE 1

Amplicons, primer pairs, and probe pairs for
identifying and quantifying SNPs in HBV.

| | SEQ | | | Product | Tm (Melting |
|---|---|---|---|---|---|
| | ID | | Position | Size | Temperature) |
| Amplicon | NO. | Sequences (5′-3′) | (nt) | (bp) | (° C.) |

| | | | | | |
|---|---|---|---|---|---|
| Set 1 | | | | | |
| Forward primer | 1 | 5′-GCATGCGTGGAACCTTTGTG-3′ | 1232-1251 | 368 | Genotype B 57.7 |
| Reverse primer | 2 | 5′-CAGAGGTGAAGCGAAGTGC-3′ | 1599-1581 | | Genotype C 66.3 |
| Anchor probe | 4 | FLU-5′-CGGCGCTGAATCCCGCGGAC-3′-P | 1436-1455 | | ΔTm = 8.6 |
| Sensor probe | 3 | 5′-ACGTCCTTTGTCTACGTCCCG-LC-Red 640-3′ | 1414-1434 | | ±30% ΔTm = ±2.5 |
| SNP site | | C/T, nt 1425 | | | |
| Set 2 | | | | | |
| Forward primer | 9 | 5′-CCGATCCATACTGCGGAAC-3′ | 1261-1279 | 340 | Genotype B 60.9 |
| Reverse primer | 10 | 5′-GCAGAGGTGAAGCGAAGTGCA-3′ | 1600-1580 | | Genotype C 54.8 |
| Anchor probe | 12 | FLU-5′-TCTGTGCCTTCTCATCTGCCGGACC-3′-P | 1552-1576 | | ΔTm = 6.1 |
| Sensor probe | 11 | 5′-TCTTTACGCGGACTCCCC-LC-Red 640-3′ | 1533-1550 | | ±30% ΔTm = ±1.8 |
| SNP site | | A/T, nt 1544 | | | |
| Set 3 | | | | | |
| Forward primer | 5 | 5′-TCATCCTCAGGCCATGCA-3′ | 3192-3209 | 416 | Genotype B 64.3 |
| Reverse primer | 6 | 5′-AACGCCGCAGACACATCCA-3′ | 392-374 | | Genotype C 46.8 |
| Anchor probe | 8 | FLU-5′-GAAAATTGAGAGAAGTCCACCACGAGTCTA-3′-P | 278-249 | | ΔTm = 16.3 |
| Sensor probe | 7 | 5′-AAGACACACGGGTGTTTCCCC-LC-Red 640-3′ | 301-281 | | ±30% ΔTm = ±4.9 |
| SNP sites | | A/G, nt 285; A/G, nt 287; G/A, nt 292; T/C, nt 294 | | | |

1. The nucleotide (nt) position number stand for a genomic position in the sequence of HBV subtype ayr (GenBank accession no. NC_003977).
2. P: the 3′ end of the anchor probe was phosphorylated to prevent probe elongation during PCR.
3. FLU: fluorescein; LC-Red 640: LightCycler-Red 640.
4. The Tm values shown here are mean values, the Tm ± 1° C. should be allowed for genotyping.
5. SNP sits are bold and underlined.

Amplicon 1 contains 1 SNP at nt 1425 with a C/T polymorphism. Amplicon 2 contains 1 SNP at nt 1544 with an A/T polymorphism. These two SNPs are located in the HBx gene. Amplicon 3 contains 4 SNPs at HBV nt 285 (A/G polymorphism), nt 287 (G/A polymorphism), nt 292 (G/A polymorphism), and nt 294 (T/C polymorphism). These 4 SNPs are located in the HBs gene.

The primers and probes were synthesized by TIB MOLBIOL. The second (or anchor) probes were labeled with fluorescein at the 3′ end; and first (or sensor) probes covering the SNPs were labeled with a LC-Red 640 dye at the 5′ end. The 3′ ends of the sensor probes were also phosphorylated.

To confirm the presence of the SNPs in the amplicons, serum samples were collected from 40 hepatitis B patients, and DNA prepared in the same manner as described below in the “HBV DNA preparation” section. After amplifying with conventional PCR reactions, all of the resultant amplicons were sequenced using ABI PRISM Big-dye kits and analyzed via an ABI 3100 Genetics Analyzer (Applied Biosystems, Foster City, Calif.). Amplicons from 20 samples were identified to contain SNPs characteristic of genotype C HBV, and amplicons from the other 20 samples contained SNPs characteristic of genotype B.

HBV DNA Preparation

Serum samples were collected from 114 chronic hepatitis B Han Chinese patients. All of the patients had been followed by the outpatient clinics at National Taiwan University Hospital. To confirm the HBV infection, the samples were tested for the presence of HBsAg, anti-HBs, anti-HBc Igs, HBeAg, and anti-HBeAg using commercially available kits (Ausab, Ausria II, Murex HBeAg/anti-HBe, Abbott Laboratories, North Chicago, Ill.). The serum HBV DNAs were also analyzed by the branched chain DNA method (QUANTIPLEX tm HBV DNA Assay, Chiron Corporation, Emeryville, Calif.) according to the manufacturer's direction. All of above procedures conformed to the ethical guidelines of the 1975 Declaration of Helsinki.

HBV genomic DNA was then prepared from the samples described above using a High Pure Viral Nucleic Acid Kit (Roche Diagnostics Applied Science, Mannheim Germany). Briefly, 200 μL aliquot of serum sample from each patient was incubated with 200 μL of a binding buffer (containing 6M guanidine-HCl, 10 mM urea, 10 mM Tris-HCl, 20% Triton X-100 (vol/vol), 200 μg of poly (A), and 0.8 mg of proteinase K) at 72° C. for 10 min. The sample was then mixed with 100 μL of isopropanol, and loaded onto glass fibers pre-packed in a High Pure filter tube. After washing twice with an Inhibitor Removal Buffer (containing 100% ethanol, 20 mmol/L NaCl, and 2 mmol/L Tris-HCl), viral nucleic acid was recovered by eluting with 100 μL of H₂O.

HBV DNA thus prepared was genotyped by traditional methods including PCR-RFLP, PCR with type-specific primers, and/or direct sequencing. 60 of the patient were identified to have genotype B HBV, and 46 of them were identified to have genotype C HBV. The HBVs from the other 8 patients could not be genotyped by these conventional methods.

Quantification of HBV

To quantify HBV, a copy-number standard curve was generated using plasmid pHBV 48. The plasmid was constructed by cloning a 1.5 mer of HBV (subtype adw1) genomic DNA fragment (nt 2851-3182/1-3182/1-1281) into the pGEM-3Z vector 8. The resultant plasmid was purified with a plasmid purification kit (QIAGEN GmbH, Hilden Germany), and quantified spectrophotometrically. The corresponding HBV titer (copy/mL) was then determined based on the mass per plasmid. The plasmid was serially diluted to obtain 10 samples with corresponding HBV titers ranging from 1×10² to 1×10¹¹ copy/mL. These 10 samples were used to generate the standard curve as described below.

2 μL of each sample were, respectively, mixed with 0.5 μL of LightCycler FastStart DNA Master Hybridization Mixture (containing Taq DNA polymerase, PCR reaction buffer, 10 mM MgCl₂, and dNTP mixture, Roche Diagnostics Applied Science, Mannheim Germany), 0.2 μL of 25 mM MgCl₂, and the set 2 primers and probes described above in the “Design of probes and primers” section. The final volume was adjusted to 5 μl, so that the concentration for each primer was 5 μM, and that for each probe was 0.5 μM. The mixture was loaded into a capillary of a LightCycler, centrifuged, and placed in the LightCycler sample carousel (Roche Diagnostics Applied Science, Mannheim Germany).

A Real-time PCR reaction was performed as follows. An initial hot start to denature DNA was carried out at 95° C. for 10 minutes, which was followed by 55 cycles of denaturing at 95° C. for 5 seconds, annealing at 55° C. for 10 seconds, and extending at 72° C. for 20 seconds. The programmed temperature transition rate was 20° C./s for the denaturing/annealing transition and 5° C./s for the annealing/extension transition. Fluorescence emitted by LC-RED640 was monitored at the end of each annealing phase. Cp values of all samples were determined and plotted against the corresponding log concentrations of the samples to create a standard curve using the LightCycler software version 3.5. The standard curve exhibited a linear range from 10² to 10¹¹ copies/mL, indicating a detection limit of 10² copies/mL.

This standard curve was then tested for quantifying HBV DNA. Test samples included 15 samples (genotypes A˜F) from a HBV Genotype Panel (International Enzymes, Inc., Fallbrook, Calif.) and 4 samples from a QUANTIPLEX bDNA kit. All of the 19 test samples contained HBVs with known titers. Aliquots of these samples were subjected to the Real-time PCR, and Cp values determined in the same manner described above. The titers corresponding to the Cp values were obtained from the standard curve. For each sample, the quantification was performed 6 times (three duplications). The results indicated that the titers of all test samples were determined accurately.

The titers thus obtained via the above-mentioned method were compared with those obtained via 3 conventional methods, including NGI SuperQuant, Roche Amplicor, and Chiron Quantiplex bDNA assays. The 19 samples were quantified by the three conventional methods according to the manufacturers' manuals. Linear regression results indicated that the titers obtained via the above-mentioned method correlated significantly with those obtained via the 3 methods (gamma=0.9866, 0.9830, and 0.999, respectively). The within-run and between-run coefficients of variation were evaluated by Pearson correlation. The results (P<0.001) indicated a remarkable reproducibility of the method.

Identification of HBV

The above-described three sets of primer pairs and probes pairs were tested for differentiating between HBV genotypes B and C, which are endemically prevalent in Taiwan, China, and Japan.

10 genotype B-containing samples and 10 genotype C-containing samples were selected from those described above. The samples were subjected to the Real-time PCR using set 2 primers and probes in the same manner described above in the “Quantification of HBV” section. After the PCR amplification, the reaction was held at 95° C. for 60 seconds, cooled to 45° C. with a transition rate of 0.5° C./s, held at 45° C. for 120 seconds, and heated to 80° C. at a transition rate of 0.5° C./s. Meanwhile, fluorescence 640 nm was monitored. After melting curves were generated for all samples, melting peaks were located by plotting the negative derivative of the fluorescence intensity with respect to temperature (−dF/dT) against temperature (T) using the LightCycler analysis software 3.5.

On the resultant plots, i.e., the first derivative melting curves, the melting peaks of all samples fell into two distinct clusters. The mean temperatures of the two clusters were characteristic of genotype B and C HBV (60.9° C. and 54.8° C., respectively). All of the 10 genotype B HBVs had Tms within the range 60.9±1.8° C. (i.e., ±30% of the ΔTm 6.1° C.), and all of the 10 genotype C HBVs had Tms within the range 54.8±1.8° C. Accordingly, 1.8° C. (or 30% of the ΔTm) was chosen as a cut-off value for differentiating between genotypes B and C. Similarly, the cut-off values (±2.5° C. and ±4.9° C.) using sets 1 and 3 amplicons and corresponding primers and probes were respectively determined. The means Tms and cut-off values were summarized in Table 1.

Then, the three sets of primer pairs and probe pairs were used to genotype all of the 60 genotype B and 46 genotype C HBVs described above in the “HBV DNA preparation” section. When using set 1 primer pairs and probe pairs, 103 of these 106 HBVs were genotyped correctly. Among the other three samples, one was not identified correctly, and two could not be identified unequivocally. When using sets 2 and 3 respectively, 1 and 2 HBVs could not be genotyped unequivocally. Nonetheless, after considering the results from any two of the three sets, all of the 106 HBVs were genotyped correctly.

As mentioned above in the “HBV DNA preparation” section, HBVs from the 8 patients could not be genotyped by conventional methods. These 8 HBVs were genotyped using the three sets of primer pairs and probe pairs. All of these HBVs were genotyped unequivocally. Direct sequencing further confirmed the genotyping results were correct. These results indicate that the genotyping method described herein is, unexpectedly, more accurate than the conventional HBV genotyping methods.

Simultaneous Quantification and Identification of HBV

HBVs in samples containing both genotypes B and C were genotyped and quantified simultaneously using the primer pairs and probe pairs and the method described above. Plasmids containing the genomes of genotypes B and C were obtained from National Taiwan University Hospital (Taipei, Taiwan). The genotypes B and C plasmids were mixed at ratios ranging from 10:1 to 1:10. The mixtures, with total titers of 10⁷ plasmids/ml, were genotyped in the same manner described above the “Identification of HBV” section. The resultant first derivative melting curves showed melting peaks and Tms characteristic of genotypes B and C. Meanwhile, the Cp value of sample was found and the titer of plasmid in the sample determined in the same manner described above in the “Quantification of HBV” section. The results indicated that one can simultaneously genotype and quantify both a major and a minor HBV populations in a mixture. The detectable titer of the minor population can be as low as 10% of that of the major population. This one-tube method is unexpectedly efficient, accurate, and sensitive for simultaneously quantifying and identifying a SNP-containing nucleic acid.

Besides genotypes B and C, this method can also be used to identify other HBV genotypes. All of the 175 HBV DNA sequences mentioned above in the “Design of probes and primers” section were aligned in the same manner as that described in the same section. The primers and anchor probe were found conserved among the genotypes A-G. SNPs in corresponding amplicons were examined. The sequence variations and corresponding frequencies were summarized in Table 2.

TABLE 2
SNP sequence variations in HBV genotypes A˜G

| | Sensor SNP | | | | | |
|---|---|---|---|---|---|---|
| | Set1 | Set2 | Set3 | | | |
| Genotype (no.) | C | A | A | A | G | T |
| A (17) | T₍₁₆₎/C₍₁₎ | T₍₁₇₎ | G₍₁₇₎ | T₍₁₃₎/G₍₃₎/C₍₁₎ | G₍₁₇₎ | T₍₁₇₎ |
| B (47) | T₍₄₂₎/C₍₅₎ | A₍₄₆₎/T₍₁₎ | G₍₃₄₎/A₍₁₂₎/T₍₁₎ | A₍₄₄₎/C₍₃₎ | G₍₄₇₎ | T₍₄₅₎/A₍₁₎/G₍₁₎ |
| C (49) | C₍₃₇₎/T₍₁₂₎ | T₍₄₅₎/A₍₂₎ | G₍₄₆₎/A₍₃₎ | G₍₄₈₎/A₍₁₎ | A₍₄₇₎/G₍₂₎ | C₍₄₃₎/A₍₅₎/G₍₁₎ |
| D (24) | T₍₂₂₎/C₍₂₎ | A₍₂₂₎/T₍₁₎/G₍₁₎ | G₍₂₄₎ | A₍₂₄₎ | G₍₂₄₎ | T₍₂₄₎ |
| E (2) | C₍₂₎ | T₍₂₎ | G₍₂₎ | G₍₂₎ | G₍₂₎ | T₍₂₎ |
| F (28) | T₍₂₅₎/C₍₃₎ | A₍₂₂₎/T₍₂₎/C₍₄₎ | G₍₂₅₎/T₍₃₎ | C₍₂₂₎/A₍₄₎/G₍₂₎ | G₍₂₇₎/A₍₁₎ | T₍₁₈₎/G₍₁₀₎ |
| G (8) | T₍₈₎ | T₍₈₎ | G₍₈₎ | G₍₈₎ | G₍₈₎ | T₍₈₎ |

The underlined characters and numbers indicated the minor sequence variations and frequencies.

As shown in Table 2, most of the 7 genotypes (except genotypes B and D) had distinct SNP combinations in the three amplicons. HBVs, therefore, can be genotyped using the combination of the three sets primer pairs and probe pairs according to steps below:

(1) determining whether HBVs that are tested belong to genotypes A, C, E, and G (Group 1) or genotypes B, D, and F (Group 2) by using set 2 primers and probes;

(2) determining whether those of Group 1 belong to genotypes A and G or genotypes C and E by using set 1 primers and probes;

(3) differentiating between genotypes A and G, and between C and E by using set 3 primes and probes;

(4) determining whether those of Group 2 belong to genotype F or genotypes B and D by using set 3 primes and probes.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A pair of primers for amplifying a target nucleic acid of Hepatitis B Virus, comprising, respectively, the sequences of SEQ ID NOs: 13 and 14, SEQ ID NOs: 17 and 14, or SEQ ID NOs: 20 and 6, wherein each primer is 8-50 nucleotides in length.

2. The pair of primers of claim 1, wherein each primer is 15-40 nucleotides in length.

3. The pair of primers of claim 2, wherein each primer is 18-30 nucleotides in length.

4. The pair of primers of claim 1, the pair of primers comprise, respectively, the sequences of SEQ ID NOs: 9 and 10, SEQ ID NOs: 1 and 2, or SEQ ID NOs: 5 and 6.

5. A pair of probes for identifying a single nucleotide polymorphism in a target nucleic acid of Hepatitis B Virus, comprising, respectively, the sequences of SEQ ID NOs: 18 and 19, SEQ ID NOs: 15 and 16, or SEQ ID NOs: 21 and 22, wherein each probe is 9-50 nucleotides in length.

6. The pair of probes of claim 5, wherein each probe is 15-40 nucleotides in length.

7. The pair of probes of claim 6, wherein each probe is 18-30 nucleotides in length.

8. The pair of probes of claim 5, wherein the pair of probes comprise, respectively, the sequences of SEQ ID NOs: 3 and 4, SEQ ID NOs: 11 and 12, or SEQ ID NOs: 7 and 8.

9. A nucleic acid obtained from amplification of a Hepatitis B Virus nucleic acid template, comprising a sequence selected from the group consisting of SEQ ID NOs: 15, 19, and 22, or a sequence complementary thereto, wherein the nucleic acid is 100-1,000 nucleotides in length.

10. The nucleic acid of claim 9, wherein the nucleic acid is 200-700 nucleotides in length.

11. The nucleic acid of claim 10, wherein the nucleic acid is 300-500 nucleotides in length.

12. A kit for simultaneously identifying a single nucleotide polymorphism in a target nucleic acid of Hepatitis B Virus and quantifying the target nucleic acid, comprising a pair of primes of claim 1 and a pair of probes of claim 2.

13. The kit of claim 12, further comprising a second pair of primers of claim 1.

14. The kit of claim 13, further comprising a third pair of primers of claim 1.

15. The kit of claim 12, further comprising a second pair of probes of claim 2.

16. The kit of claim 15, further comprising a third pair of probes of claim 2.

17. A method for simultaneously identifying a single nucleotide polymorphism (SNP) in a target nucleic acid from a Hepatitis B Virus (HBV) and quantifying the target nucleic acid, comprising:
providing a first probe that is identical or complementary to a first sequence of the target nucleic acid that covers a base corresponding to the SNP; and a second probe that is identical or complementary to a second sequence of the target nucleic acid that does not cover the base corresponding to the SNP;
amplifying, by a polymerase chain reaction with a pair of primers, the target nucleic acid to form a double-stranded nucleic acid product containing the first sequence and the second sequence;
hybridizing to the nucleic acid product in a solution to form a first duplex with the first probe that is covalently bounded to a first fluorophore and to form a second duplex with the second probe that is covalently bounded to a second fluorophore, one of the first and second fluorophores being a donor fluorophore and the other being an acceptor fluorophore, so that, when the first probe and the second probe are hybridized to the nucleic acid product, the donor fluorophore and the acceptor fluorophore are in close proximity to allow fluorescence resonance energy transfer therebetween;
heating the solution to an elevated temperature that is above the melting temperature of the duplex formed by the first probe and its complementary sequence;
identifying the SNP by monitoring fluorescent emission change of the acceptor fluorophore upon irradiation of the donor fluorophore with an excitation light, the change being a function of the elevated temperature; and
quantifying the target nucleic acid by monitoring the fluorescent emission of the acceptor fluorophore.

18. The method of claim 17, wherein the quantifying step is conducted by comparing the intensity of the fluorescent emission to a predetermined value.

19. The method of claim 18, wherein the first and second probes hybridize to the same strand of the nucleic acid product.

20. The method of claim 17, wherein the first and second probes hybridize to the same strand of the nucleic acid product.

21. The method of claim 20, wherein the identifying step is conducted by
generating a first derivative melting curve of the first duplex;
determining a temperature value corresponding to a melting peak on the curve; and
comparing the temperature value with the melting temperature of the duplex formed by the first probe and its complementary sequence,
whereby a single nucleotide polymorphism in the target nucleic acid is present when the temperature value is lower than the melting temperature and is absent when the temperature value is the same as the melting temperature.

22. The method of claim 17, wherein the identifying step is conducted by
generating a first derivative melting curve of the first duplex;
determining a temperature value corresponding to a melting peak on the curve; and
comparing the temperature value with the melting temperature of the duplex formed by the first probe and its complementary sequence,
whereby a single nucleotide polymorphism in the target nucleic acid is present when the temperature value is lower than the melting temperature and is absent when the temperature value is the same as the melting temperature.

23. The method of claim 22, wherein the quantifying step is conducted by comparing the intensity of the fluorescent emission to a predetermined value.

24. The method of claim 23, wherein the first and second probes hybridize to the same strand of the nucleic acid product.

25. The method of claim 17, wherein the pair of probes comprise, respectively, the sequences of SEQ ID NOs: 15 and 16, SEQ ID NOs: 18 and 19, or SEQ ID NOs: 21 and 22.

26. The method of claim 25, wherein the corresponding pair of primers comprise, respectively, the sequences of SEQ ID NOs: 13 and 14, SEQ ID NOs: 17 and 14, or SEQ ID NOs: 20 and 6.

27. The method of claim 26, wherein the identifying step is conducted by
generating a first derivative melting curve of the first duplex;
determining a temperature value corresponding to a melting peak on the curve; and
comparing the temperature value with the melting temperature of the duplex formed by the first probe and its complementary sequence,
whereby a single nucleotide polymorphism in the target nucleic acid is present when the temperature value is lower than the melting temperature and is absent when the temperature value is the same as the melting temperature.

28. The method of claim 27, wherein the quantifying step is conducted by comparing the intensity of the fluorescent emission to a predetermined value.

29. The method of claim 28, wherein the first and second probes hybridize to the same strand of the nucleic acid product.


SEQUENCE LISTING (22 sequences)

SEQ ID NO: 1 - DNA, 20 bp, Artificial Sequence
  primer
gcatgcgtggaacctttgtg

SEQ ID NO: 2 - DNA, 19 bp, Artificial Sequence
  primer
cagaggtgaagcgaagtgc

SEQ ID NO: 3 - DNA, 21 bp, Artificial Sequence
  Sensor probe
acgtcctttgtctacgtcccg

SEQ ID NO: 4 - DNA, 20 bp, Artificial Sequence
  Anchor probe
cggcgctgaatcccgcggac

SEQ ID NO: 5 - DNA, 18 bp, Artificial Sequence
  primer
tcatcctcaggccatgca

SEQ ID NO: 6 - DNA, 19 bp, Artificial Sequence
  Primer
aacgccgcagacacatcca

SEQ ID NO: 7 - DNA, 21 bp, Artificial Sequence
  Sensor probe
aagacacacgggtgtttcccc

SEQ ID NO: 8 - DNA, 30 bp, Artificial Sequence
  Anchor probe
gaaaattgagagaagtccaccacgagtcta

SEQ ID NO: 9 - DNA, 19 bp, Artificial Sequence
  Primer
ccgatccatactgcggaac

SEQ ID NO: 10 - DNA, 21 bp, Artificial Sequence
  Primer
gcagaggtgaagcgaagtgca

SEQ ID NO: 11 - DNA, 18 bp, Artificial Sequence
  Sensor probe
tctttacgcggactcccc

SEQ ID NO: 12 - DNA, 25 bp, Artificial Sequence
  Anchor probe
tctgtgccttctcatctgccggacc

SEQ ID NO: 13 - DNA, 8 bp, Artificial Sequence
  Primer
tactgcgg

SEQ ID NO: 14 - DNA, 10 bp, Artificial Sequence
  Primer
ggtgaagcga

SEQ ID NO: 15 - DNA, 11 bp, Artificial Sequence
  Sensor probe
tacgcggactc

SEQ ID NO: 16 - DNA, 11 bp, Artificial Sequence
  Anchor probe
gccttctcatc

SEQ ID NO: 17 - DNA, 9 bp, Artificial Sequence
  Primer
cgtggaacc

SEQ ID NO: 18 - DNA, 9 bp, Artificial Sequence
  Probe
ttgtctacg

SEQ ID NO: 19 - DNA, 9 bp, Artificial Sequence
  Probe
cgctgaatc

SEQ ID NO: 20 - DNA, 9 bp, Artificial Sequence
  Primer
ctcaggcca

SEQ ID NO: 21 - DNA, 14 bp, Artificial Sequence
  Sensor probe
acacgggtgtttcc

SEQ ID NO: 22 - DNA, 10 bp, Artificial Sequence
  Anchor probe
attgagagaa